(19) United States
(12) Patent Application Publication
(10) Pub. No.: US 2013/0332286 A1
(43) Pub. Date: Dec. 12, 2013

(54) ACTIVITY TYPE DETECTION AND TARGETED ADVERTISING SYSTEM

(71) Applicants: Pedro J. Medelius, Merritt Island, FL (US); Espen D. Kateraas, Aliso Viejo, CA (US)

(72) Inventors: Pedro J. Medelius, Merritt Island, FL (US); Espen D. Kateraas, Aliso Viejo, CA (US)

(21) Appl. No.: 14/000,797

(22) PCT Filed: Feb. 21, 2012
(86) PCT No.: PCT/US12/25900

§ 371 Date: Aug. 21, 2013

Related U.S. Application Data
(60) Provisional application No. 61/445,212, filed on Feb. 22, 2011.

(51) Int. Cl. G06Q 30/02; A61B 5/11

(52) U.S. Cl. CPC G06Q 30/0269; A61B 5/1118; A61B 5/1126
USPC 705/14.66; 702/141; 702/131

(57) ABSTRACT

A physical activity data collection system may include one or more accelerometers configured to provide an output indicative of movements of an individual. The system may also include a controller configured to analyze information relating to the movements of the individual, based on the output of the one or more accelerometers; determine a physical activity type associated with the analyzed information; and store the determined physical activity type in a memory.

This application claims priority to U.S. Provisional Patent Application No. 61/445,212, filed Feb. 22, 2011, the entire contents of which are incorporated herein by reference.

### TECHNICAL FIELD

Embodiments of the present invention may include an apparatus to monitor physical activity of an individual and determine a particular type of activity in which the individual is engaged. Advertising may be selected and directed to the individual based on this information.

### BACKGROUND

Individuals that participate in certain types of activities often purchase equipment or articles related to that participation. For example, a runner may make regular purchases of athletic shoes and clothing, exercise monitoring equipment, sports drinks and nutrition products, etc. Similar patterns of purchasing behavior may exist for many types of activities, including cycling, swimming, skiing, walking, parachuting, horseback riding, etc. Targeted advertising to such groups of individuals, based, e.g., on detected participation in a certain type of activity, may increase the effectiveness and efficiency of advertising efforts.

### SUMMARY OF THE INVENTION

One aspect of the disclosure a physical activity data collection system. The system may include one or more accelerometers configured to provide an output indicative of movements of an individual. The system may also include a controller configured to analyze information relating to the movements of the individual, based on the output of the one or more accelerometers; determine a physical activity type associated with the analyzed information; and store the determined physical activity type in a memory.

Some embodiments may also include a method of identifying a type of physical activity in which an individual engages. The method may include monitoring outputs of one or more accelerometers configured to provide an output indicative of movements of the individual; analyzing information relating to the movements of the user, based on the output of the one or more accelerometers; determining a physical activity type associated with the analyzed information; and storing the determined physical activity type in a memory.

### BRIEF DESCRIPTION OF THE DRAWINGS

FIG. 1 is a diagrammatic representation of a data collection unit according to an exemplary disclosed embodiment.

FIG. 2 is a functional block level diagram of a data collection unit according to an exemplary disclosed embodiment.

FIG. 3 is a diagrammatic representation of a data collection unit according to an exemplary disclosed embodiment.

FIGS. 4A and 4B are diagrammatic representations of closure systems for a data collection unit according to exemplary disclosed embodiments.

FIG. 5 is a diagrammatic representation of a closure system for a data collection unit according to an exemplary disclosed embodiment.

FIG. 6 is a diagrammatic representation of a closure system for a data collection unit according to an exemplary disclosed embodiment.

FIG. 7 is a diagrammatic representation of a physical activity monitoring system according to an exemplary disclosed embodiment.

FIG. 8 is a block diagram representation of a data collection unit according to an exemplary disclosed embodiment.

### DETAILED DESCRIPTION

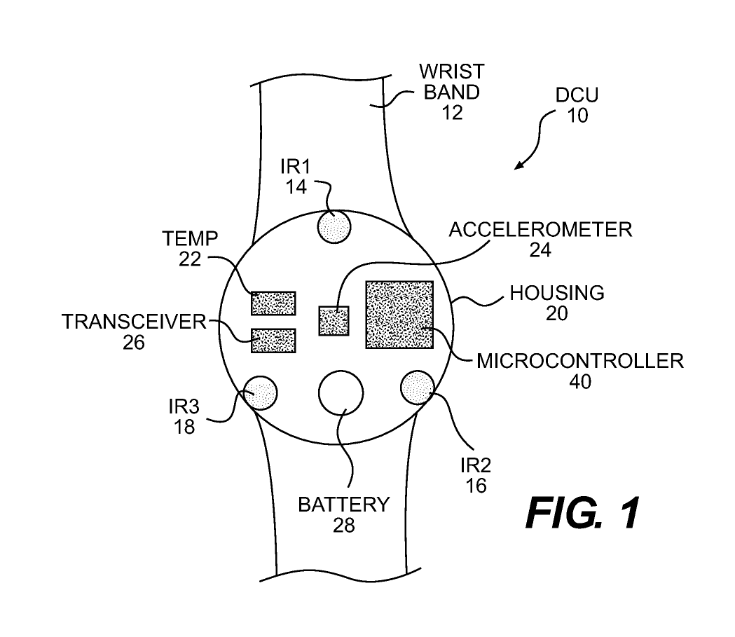

FIG. 1 provides diagrammatic representation of a data collection unit according to an exemplary disclosed embodiment. As illustrated in FIG. 1, the disclosed data collection unit 10 may be configured as a wearable article. In certain embodiments, for example, the data collection unit may be incorporated into an article wearable on an individual's wrist. Such an article would offer the advantage of being minimally intrusive, as most people are accustomed to wearing articles fastened to the wrist. The wrist unit could be fashioned as a simple wrist band stylized in various colors and patterns. The band may be adjustable, shockproof, and secured to the wrist using a hook and loop closure, a buckle closure, an elastic material requiring no separate closure device, or with any other suitable fastening configuration. The band can be made from various materials including, for example, a waterproof material, neoprene, polymer, nylon, leather, metal, or any other wearable material.

In one embodiment, data collection unit 10 may be embedded into a small, self-contained wrist band 12. In such a configuration, there may be little or no external indication of the presence of the hardware components of the data collection unit. In other embodiments, the data collection unit may be incorporated into a watch, bracelet, heart rate monitor or other wearable article to provide added functionality to those devices. In addition to the wrist, the disclosed data collection unit may be positioned over any portion of a user's body (e.g., the neck, chest, ankle, head, or thigh) that can provide suitable access to the biological markers needed for monitoring the user's level of physical exertion. For example, the data collection unit may be configured as or incorporated into shoe soles, ear clips, a necklace, ankle band, sock, belt, glove, ring, sunglasses, hat, and/or a headband.

Data collection unit 10 includes a sensor array (including one or more sensors) configured to monitor biological markers that vary with the level of exertion of an individual. The monitored biological markers may include, for example, pulse rate, body temperature, blood oxygen content, or any other suitable marker. Within the sensor array, each sensor may be configured to monitor only a single biological marker. Alternatively, an individual sensor in the array may be configured to monitor multiple biological markers.

In one embodiment, data collection unit 10 may include several sensors. These sensors may include any arrangement of one or more sensors capable of monitoring biological characteristics and/or movement associated with a user of data collection unit 10. In one exemplary embodiment, as shown in FIG. 1, data collection unit 10 may include at least one infrared sensor 14, a temperature sensor 22, and/or an accelerometer 24.

In the exemplary embodiment shown in FIG. 1, data collection unit 10 includes three infrared sensors 14, 16, 18. Suppliers of appropriate infrared transmitter/receivers include Vishay Semiconductors, among others.

Each infrared sensor may be configured as a transmitter/receiver capable of monitoring the oxygen content of blood passing through nearby blood vessels. Specifically, each infrared sensor can be configured to both emit infrared radiation into the body of the wearer of data collection unit 10 and detect the level of infrared radiation received at the sensor. The wavelength of the emitted radiation can be selected according to the requirements of a particular application. In one embodiment, infrared sensors 14, 16, and 18 can be configured to emit infrared radiation in a wavelength range of about 650 nm to about 950 nm.

The difference between the emitted radiation level and the detected radiation level is characteristic of the amount of infrared radiation absorbed by the body and, especially, by oxygen-carrying blood. This sensed absorption level can be used to determine the pulse rate of the wearer of data collection unit 10. Particularly, the infrared absorption level may be affected by the expansion and contraction of nearby blood vessels and the oxygen content of blood passing through nearby vessels, which are both physical characteristics that vary together with heart rate. Thus, the rate of observed changes in infrared absorption characteristics of the body can enable a calculation of the wearer's heart rate.

While only one infrared sensor may be needed depending on the functional requirements of a particular embodiment, including two or more infrared sensors, or even three or more infrared sensors, can serve to increase the reliability of the data collected from these sensors. As illustrated in FIG. 1, infrared sensors 14, 16, and 18 may be spaced apart from one another. In certain embodiments, these sensors may be located along a perimeter of a central housing 20 of data collection unit 10. Spacing infrared sensors 14, 16, and 18 apart from one another can maximize the possibility that at least one sensor contacts the wearer's skin at all times, even during the movements associated with physical activities.

A power management scheme may be employed to lower the power requirements of infrared sensors 14, 16, and 18. For example, the transmitter portion of each sensor may be pulsed at a predetermined duty cycle to conform to the power specifications of a particular configuration. In one exemplary embodiment, the infrared transmitters of sensors 14, 16, and 18 can be pulsed using a 1% duty cycle at a rate of about 8 pulses per second.

Data collection unit 10 may also include a temperature sensor 22. Temperature sensor 22 may be configured to monitor the body temperature of the wearer of data collection unit 10 by measuring the temperature outside of housing 20 and, for example, against the skin of the wearer. Additionally, temperature sensor 22 may be configured to measure the temperature inside housing 20. Using the difference between the temperature measurements from inside and outside of housing 20, it can be determined whether an observed temperature change outside of the housing is likely attributable to atmospheric conditions or an actual change in body temperature of the wearer of data collection unit 10. While certain embodiments may include only one temperature sensor, other embodiments may include multiple temperature sensors in order to meet a desired set of operational characteristics (e.g., monitoring body temperature from multiple locations on data collection unit 10; separate temperature sensors to monitor the temperature inside and outside of housing 20; etc.).

Temperature sensor 22 may include any suitable device for ascertaining the body temperature of an individual. For example, temperature sensor 22 may include a digital or analog device and may include thermocouples, diodes, resistance temperature detectors (RTDs), or infrared detectors. Suitable temperature sensors may be obtained from various suppliers, including Analog Devices Inc., Omega, or Texas Instruments. For certain types of temperature sensors, contact with the individual's skin may aid in obtaining accurate body temperature measurements. On the other hand, in certain instances where, for example, infrared sensors provide the primary mode of measuring body temperature, mere proximity to the individual's skin may be sufficient to accurately determine body temperature of the user.

Additionally, data collection unit 10 may include an accelerometer 24 to monitor motion of data collection unit 10. In certain embodiments, accelerometer 24 includes only a single axis accelerometer configured to detect motion along one axis. Other embodiments, however, may include multiple accelerometers. In one exemplary embodiment, accelerometer 24 may include a three-axis accelerometer, which includes three accelerometers arranged orthogonally with respect to one another. With such an arrangement, accelerometer 24 may be able to detect or monitor movements along three separate axes.

A three-axis accelerometer may be especially useful for the detection of movements associated with exercise and certain types of physical activity. Generally, most sports or types of physical activity produce a signature pattern of movements that can be detected using an accelerometer. In this way, accelerometer 24 can help confirm whether the wearer of data collection unit 10 is engaged in physical activity and, in certain cases, can help determine the type of sport or activity in which the wearer is engaged.

Other embodiments of data collection unit 10 may include additional or different sensors. For example, data collection unit 10 may include a carbon dioxide detector, additional accelerometers, a breathing rate sensor, or any other type of sensor suitable for monitoring physical activity levels.

In addition to the infrared sensors described above, the pulse of the wearer of data collection unit 10 may be ascertained using any other type of sensor suitable for monitoring the wearer's heart rate. In one embodiment, for example, electro-cardiogram based technology may be incorporated into data collection unit 10.

Data collection unit 10 may also include a transceiver 26 for establishing communication with devices external to data collection unit 10. To address power requirements, data collection unit 10 may also include a battery 28.

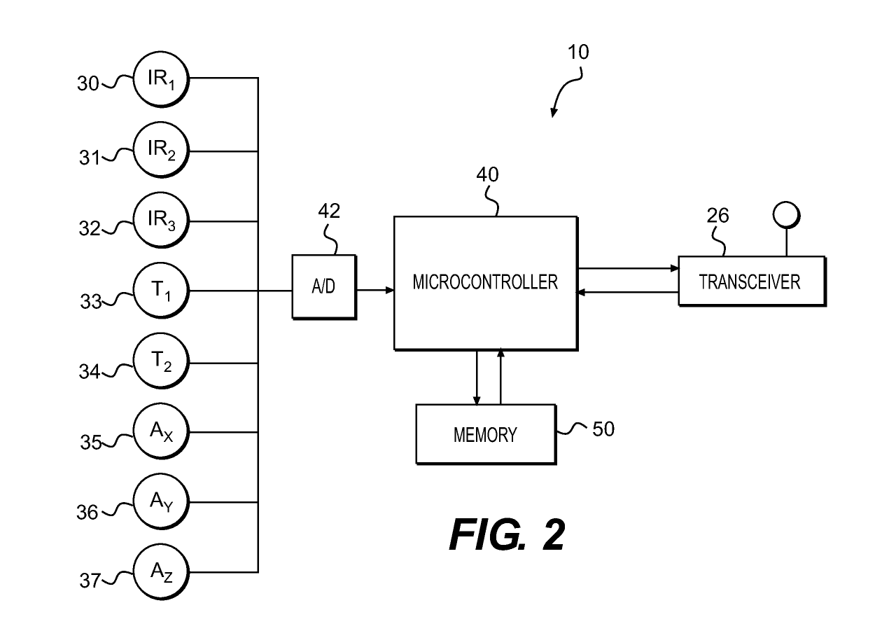

FIG. 2 provides a schematic, functional block level diagram of data collection unit 10, according to an exemplary disclosed embodiment. Within data collection unit 10, several sensed quantities can be provided to a microcontroller 40 for processing. For example, these sensed quantities may include outputs 30, 31, and 32 from infrared sensors 14, 16, and 18, respectively. Additionally, these sensed quantities may include temperature sensor outputs 33 and 34. Temperature output 33 may correspond to the temperature inside housing 20, for example, and temperature output 34 may correspond to the observed temperature outside of housing 20. The sensed quantities may also include accelerometer outputs 35, 36, and 37, each corresponding to a unique axis of movement.

Microcontroller 40 can store the data associated with the sensed quantities in a memory 50 in raw form or, alternatively, after processing. Further, the data relating to the sensed quantities can be transmitted to a remote location by transceiver unit 26.

Any suitable microcontroller 40 may be included in data collection unit 40. In one embodiment, microcontroller 40 includes a small microcontroller having dimensions of about 0.4 inches by 0.4 inches, or smaller. One suitable microcontroller includes the PIC18F series of microcontroller manufactured by Microchip Inc. Preferably, microcontroller 40 would exhibit low power characteristics and would require from about 10 microamps to about 50 microamps during normal operation and between 5 milliamps to about 20 milliamps while transmitting data.

Microcontroller 40 of data collection unit 10 has several responsibilities. Among these responsibilities, microcontroller 40 periodically collects data from the available sensors via an analog-to-digital converter 42. The frequency of data collection can be selected to meet the requirements of a particular application. In one embodiment, microcontroller 40 may sample the data from the sensors at least once per second. Higher or lower sampling frequencies, however, may also be possible.

Microcontroller 40 may be configured with the ability for selecting from among multiple data sampling frequencies depending on sensed conditions. For example, microcontroller 40 may be programmed to sample the sensor outputs slower than once per second (e.g., once per every 10 seconds) when microcontroller 40 determines that the user of the device is at rest or at a normal level of physical exertion. Similarly, microcontroller 40 may be configured to sample the sensor outputs more frequently (e.g., at least once per second) when the user's physical exertion level exceeds a predetermined threshold. In certain embodiments, and during periods of physical exertion, microcontroller 40 may collect sensor data up to five times per second, ten times per second, or even more, to ensure that rapidly changing quantities such as pulse rate and blood oxygen, which may cycle on the order of 200 times per minute during periods of extreme physical exertion, can be accurately evaluated.

When appropriate, microcontroller 40 may also enter a rest state to conserve power. For example, when infrared sensors 14, 16, or 18 provide no pulse readings or accelerometer 24 registers no movements over a certain period of time, microcontroller 40 may determine that data collection unit 10 is not being worn. Under such conditions, microcontroller 40 may slow the sensor sampling period to once every thirty seconds, once every minute, or to another suitable sampling frequency. Additionally, microcontroller 40 may be configured to sample only a portion of the available sensors during times of physical inactivity or when data collection unit 10 is not being worn. In one embodiment, for example, once microcontroller 40 determines that the user is not wearing data collection unit 10, microcontroller 40 may begin sampling the output of temperature sensor 22 alone. In such a configuration, a perceived rapid change in temperature may indicate that data collection unit 10 is in use and may prompt the controller to “wake up” and restore full functioning data collection.

Microcontroller 40 can be configured to analyze the data collected from the sensors onboard data collection unit 10. For example, data from infrared sensors 14, 16, 18 can be used to compare the transmitted infrared signal to the received infrared signal and calculate the blood oxygen saturation level via known algorithms. Microcontroller 40 may also be configured to calculate the pulse rate by monitoring the frequency of changes in the blood oxygen saturation level.

As noted above, microcontroller 40 can be configured to store raw or processed data in memory 50 included in data collection unit 10. Memory 50 may include any suitable storage unit including, for example, a solid state non-volatile serial or parallel access memory. In certain embodiments, the memory may include a storage capacity of at least 32 MB. Suitable memory units include RAM, NVRAM, and Flash memory. It is also possible to use an internal microcontroller memory to store data, especially if microcontrollers are developed that include internal memory sizes greater than the currently available 64 kB sizes.

In the case that microcontroller 40 is configured to store raw data, microcontroller 40 may sample the outputs of the sensors onboard data collection unit 10 and simply store those values in memory 50. Those stored values can then later be downloaded from data collection unit 10 and processed using devices and/or systems external to data collection unit 10.

While it is possible to store raw data collected from the sensor devices, microcontroller 40 may also be configured to process the data sampled from the sensors of data collection unit 10 prior to storage in memory 50. For example, microcontroller 40 may be configured to calculate pulse rate, temperature, acceleration and average each calculated value over periods of up to thirty seconds, sixty seconds, or more to remove noise and enhance accuracy of the readings. Microcontroller 40 can be further configured to store these time averaged, filtered pulse rate/temperature/acceleration readings at preselected intervals (e.g., once or twice per minute). Such a scheme may conserve memory and/or power resources yet still provide useful information. These processed or conditioned data signals stored in memory, in certain cases, can even be more useful, as they may exhibit less noise and rapidly fluctuating values, which can detract from the reliability of the data.

Microcontroller 40 may be configured to condition the signals received from one or more of the sensors onboard data collection unit 10. During movement associated with physical activity, a significant amount of noise may be imparted to the signals generated by the onboard sensors. Such noise is especially prevalent in the data provided by the infrared sensors, which can be used to determine heart rate. Digital signal processing techniques may be employed to eliminate at least some of the noise from these signals and increase the accuracy of the heart rate calculation.

Microcontroller 40 may also be configured to determine when the user is at rest and when the user is exercising. In addition to using this information to control the data collection and storage rates, this information can be used, for example, in conjunction with a physical activity rewards allocation system to provide rewards-based incentives to the user of data collection unit 10. That is, the user of data collection unit 10 may receive rewards in the form of merchandise, merchandise discounts, currency, and/or free or discounted services based on the amount of time the user spends exercising and/or upon the level of physical exertion during exercise. The information may also be used to track physical activity levels for purposes of assessing the physical health of individuals. For example, the information may be tracked and used to determine the fitness, health, or well-being of private or public employees in order to provide worker incentives. Alternatively or additionally, this information could be used by the insurance industry to set rates/premiums tailored to an individual or discounted for a group of individuals participating in a physical activity tracking program.

Microcontroller 40 can be configured to determine when the user's level of activity qualifies as exercise. For example, microcontroller 40 can assimilate one or more of the user's pulse rate, temperature, and acceleration levels into a exercise evaluation score. Comparing the exercise evaluation score with a predetermined threshold level, microcontroller 40 can determine that the user is exercising when the exercise evaluation score exceeds the threshold.

The microcontroller's accuracy in determining the physical activity level or exertion level of a user can be refined according to any desired algorithm. In one embodiment, for example, microcontroller 40 may be configured to determine the relative reliability of the data provided by the sensors onboard data collection unit 10 and assign weighting factors (e.g., values between 0 and 1) to those outputs based on the perceived reliability of the data from each output. For example, if one of the infrared sensors is emitting a stable, oscillating output signal with a low noise level and another is emitting a noisy signal, then microcontroller 40 can assign a higher weighting factor to the higher quality signal and a lower weight to the noisy signal. In this way, microcontroller 40 can minimize the effects of extraneous noise and low quality data and maximize the measurement reliability when high quality data output signals are available.

Microcontroller 40 can be programmed with a common baseline threshold for use with all users of the disclosed data collection unit 10. Alternatively, microcontroller 40 may be used to calculate and periodically update a unique threshold determined for a specific user of a particular data collection unit. For example, as the user wears and uses data collection unit 10 over a period of time, microcontroller 40 may “learn” about the user by monitoring and storing quantities (e.g., heart rate, acceleration levels, and temperature) associated with periods during which the user is at rest and exercising. Using a predefined exercise threshold algorithm, the microcontroller can use this information to tailor the exercise threshold and store a new, updated exercise threshold based on the current fitness level of the user. The predefined algorithm may be loaded into the microcontroller's operating instruction set upon manufacture and may be updated via download from a central server system. It should be noted that while the present disclosure may refer at times to an exercise threshold, the disclosed methods and systems are not limited to any particular form of activity, such as exercise. Rather, the disclosed systems and methods may be used to determine, monitor, etc. any type of physical activity and an any activity level.

Ultimately, microcontroller 40 can be configured to determine when the user's level of physical activity surpasses the exercise threshold. Once the user exceeds the exercise threshold, the microcontroller may start a timer that monitors the amount of time the user spends above the exercise threshold. Further, via the sensed pulse rate, temperature, and acceleration levels measured, microcontroller 40 can determine and store a quantity that tracks the amount by which the user's physical activity exceeds the exercise threshold. This information, together or separate from exercise time, may be used by microcontroller 40 or, more preferably, a remote rewards allocation system to determine a rewards quantity accrued by the user during each period of exercise. Alternatively or additionally, this information can be used by a physical activity tracking system to determine worker incentives or to set/adjust insurance rates/premiums.

Data collection unit 10 may also include a feedback element, including, for example, a display, light, audible speaker, or other suitable sensory interface device. During periods when the user's physical activity exceeds the exercise threshold and qualifies for rewards accrual, microcontroller 40 may activate the feedback element to indicate to the user that the exercise threshold has been exceeded and rewards are being accrued. For example, an LED may be included that blinks during periods of qualifying exercise. In other embodiments, a speaker may emit an audible beep every few seconds during periods of qualifying exercise. In still other embodiments, a rewards indicator may be projected on a display during qualifying exercise sessions. Such an embodiment would be especially useful where data collection unit 10 was incorporated into a watch or other type of device including a display.

Microcontroller 40 of data collection unit 10 may be configured to control transmission of data to one or more remote locations. In one embodiment, microcontroller 40 can activate transceiver 26, as illustrated in FIG. 2, with a low duty cycle of less than about 1% to detect the presence of suitable data collection portals. A data collection portal can include any intended recipient of the data acquired by data collection unit 10. In one embodiment, a data collection portal may be associated with a physical activity rewards allocation system and may forward the data received from data collection unit 10 to a central management facility that handles the operation of the rewards system. In another embodiment, the data collection portal may be associated with a threshold exercise tracking system for purposes of determining the fitness, health, or well-being of private and public employees for worker incentives. The data collection portal may also be associated with an insurance rate/premium setting system that tailors rates or adjusts premiums based on the physical activity level of individuals and/or groups.

When data collection unit 10 detects a data collection portal (e.g., either through a wired or wireless data connection) and communication is established, download of the data will commence, for example, after proper identification of the user and of the portal has been achieved. This may prevent eavesdropping by unauthorized parties. Identification of the user may include transmission of a unique code assigned to each data collection unit and/or user of the data collection unit. A user-selectable password can be used to allow data to be downloaded by the data collection portal. In other embodiments, passive identification of a user may displace the need for password protected downloads. For example, the microcontroller may be configured to determine and store a biological signature of an authorized user of the data collection unit. Such a signature may be determined using the same array of sensors used monitor temperature, pulse rate, and acceleration levels. Alternatively, one or more additional sensors (e.g., a skin pigment sensor, pH sensor, etc.) may be included to aid in user recognition.

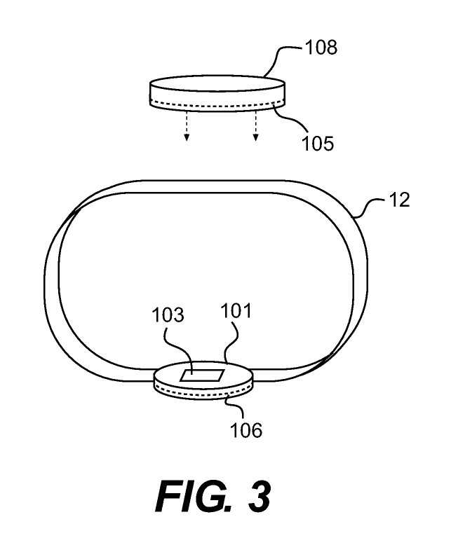

One or more other devices, including, e.g., an RFID tag may be employed to facilitate the transmission of data to a data collection portal. For example, in response to a radio frequency interrogation signal, an RFID tag located on data collection unit 10 may power on using an onboard power source, such as battery 28, or using energy provided by the interrogation signal. The RFID tag can respond to the interrogation signal by transmitting data to a location/receiver remotely located with respect to data collection unit 10. The information transmitted may include information about data collection unit 10. For example, the transmitted information may include a signature code associated with a particular data collection unit 10. Additionally, the transmitted information may include any other data that may aid in recognition of the particular data collection unit 10. Such an RFID tag may be attached or integrated with data collection unit 10 at any suitable location. For example, an RFID tag may be included in housing 20 (FIG. 1), battery holder 105, battery holder 106, cradle 108, housing 101 (FIGS. 3, 5, 6), or at any other suitable location on data collection unit 10 or along band 12.

Alternatively or additionally, an RFID tag or other similar device for transmitting data from data collection unit 10 (e.g., microcontroller 40 coupled with transceiver 26) may be used to transmit information about the user of data collection unit 10. This information can include, for example, medical emergency data, insurance information, name, home address, phone numbers, vital statistics, allergies, blood type, etc.

The transmitted information may also be used to recognize an individual wearer of data collection unit 10. For example, based on a particular piece of information (e.g., a signature code, name, address, etc.) an interrogating device or data portal may “recognize” the wearer of data collection unit 10. In response, the receiver of this information may take some action based on the recognition of the user of data collection unit 10. In certain embodiments, such information may be used to determine the location of a user of data collection unit 10; determine the frequency that the user visits a particular establishment, such as a health club, spa, pools; etc.

Data collection unit 10 may also be configured to detect potentially fraudulent use by a user. For example, because the user may receive rewards based on an indication by data collection unit 10 that the user had engaged in qualifying physical activity for a certain period of time, certain individuals may be motivated to simulate a state of physical activity, wear multiple data collection units, or engage in other types of fraudulent activity. With the robust sensor array included in data collection unit 10, the likelihood of data collection unit 10 being “fooled” by simulated physical activity is minimized.

Additionally, microcontroller 40 may be configured to generate and deliver a low power, low duty cycle pulse to metal contacts located, e.g., on the base of housing 20. These pulses may have a duration of less than about 100th of a millisecond per pulse and will be transmitted over short distances around data collection unit 10. The same metal contacts on the base of housing 20 can also serve as an antenna and can aid in detection of similar signals in close proximity. When such a signal is detected, it may indicate that a user is wearing more than one data collection unit devices. If the detected signal remains constant over a certain period of time, further suggesting that more than one data collection unit 10 is in use by a single user, then either the emitting or detecting data collection unit, or both, may be configured to shut down.

Suitable data collection portals may include those located within a predetermined distance from data collection unit 10. In certain embodiments, data collection unit 10 may be configured to transmit data to portals located within about ten feet. In other embodiments, this transmission distance may be extended up to about 50 feet.

Once transmission of data stored in data collection unit 10 commences, a handshaking process may be employed to validate the integrity of the data transmitted and to request retransmission of the data, if necessary. After data collection unit 10 establishes that the data has been successfully transmitted to the data collection portal, microcontroller 40 can delete the previously stored data.

Transmission of data to a data collection portal may also be controlled based on the availability of stored data. For example, if no new data has been stored in memory 50 since the last successful download, then microcontroller 40 may determine that there is nothing to transmit. Under these conditions, microcontroller 40 may forego searching for a suitable data collection portal within range and will leave the data collection unit transceiver 26 powered down until data is subsequently stored in memory.

Other schemes for data transmission initiation may be employed. For example, rather than the microcontroller periodically searching for a suitable data collection portal within range, microcontroller 40 may be configured to simply respond to an interrogation signal continuously or periodically emitted from a data collection portal. If microcontroller 40 receives such an interrogation and determines that the emitting data collection portal is within transmission range, then microcontroller 40 can activate transceiver 26 and commence data transmission.

Data transmission may be accomplished via any suitable scheme for transmission of data. In one embodiment, the data stored in the data collection unit may be transferred via a wired connection including a cable and cable interface. In one embodiment, data transmission can be accomplished via a USB data cable that enables charging of data collection unit 10 while data is downloaded. Data transmission may also be accomplished via a wireless connection including a radio frequency or optical transmission link. In certain embodiments, for example, data collection unit 10 can be Bluetooth or Zigbee enabled or may transmit data via an infrared optical link.

In certain embodiments, data transmission can extend beyond the limits of the onboard transceiver. For example, using a Bluetooth enabled data collection unit coupled with an external device, such as a cell phone, PDA, personal computer, etc., data can be relayed from data collection unit 10 through the external device and on to a data collection portal or even directly to the management facility.

Data collection unit 10 may include any suitable power source for meeting the power requirements of the unit. For example, data collection unit 10 may include a replaceable or rechargeable battery 28. In certain embodiments, three-volt lithium batteries contained within a 1.2 cm package may be included in data collection unit 10. Additionally, or alternatively, a solar cell may be included either alone or in combination with one or more batteries. In addition to serving as a stand alone power source, the solar cell may also function to recharge the batteries.

In another embodiment, a motion activated regeneration device may be included for purposes of powering the data collection unit and/or recharging batteries.

The sensors included in data collection unit 10 may be located together in a single housing 101, as shown in FIG. 3. In one embodiment, accelerometer 24; infrared sensors 14, 16, and 18; and/or temperature sensor 22 (and any combinations thereof) may be integrated together to form a sensor array, for example, on a common printed circuit board. While this sensor array could be located at any position along wrist band 12, in one embodiment, the sensor array is located in housing 101 located at the point along wrist band 12 that is adjacent to the underside of the wrist of a user. In this configuration, the sensor array, or portions thereof, could be made to contact the underside of the user's wrist when data collection unit 10 is worn. Housing 101 may include a window 103, fabricated from infrared transparent material, for example, to allow radiation emitted from infrared sensors 14, 16, and 18 to pass out of housing 101 and impinge upon the underside of the user's wrist. In turn, window 103 also allows infrared radiation reflected or emitted from the user's skin to pass into housing 101 via window 103.

Housing 101 can be constructed of a material different from wrist band 12. For example, housing 101 may be fabricated from a polymer, metal, rubber, or any other material suitable for a desired application. In certain embodiments, housing 101 can be constructed from a conducting material to establish an electrical or thermal conduction path, if desired, between any of the sensors of data collection unit 10 and the skin of the user.

Housing 101 can also be formed integrally with wrist band 12. In such an embodiment, housing 101 would be formed of the same material as wrist band 12 and may have the same thickness, or a slightly thicker profile, as compared to wrist band 12.

Battery 28 may include a single battery. Alternatively, battery 28 may include multiple individual batteries connected in series, in parallel, or, alternatively, configured to separately and independently provide power to various electrical components of data collection unit 10.

Battery 28 may be mounted within or adjacent to housing 101. In certain embodiments, battery 28 may be positioned in a battery holder 106 adjacent to housing 101. Battery holder 106 may be formed separately from housing 101 and may be attached to housing 101. Alternatively, battery holder 106 may be formed as an integral part (or an internal part) of housing 101.

In other embodiments battery 28 may be mounted in a holder spaced apart from housing 101. For example, a battery holder 105 may be attached to wrist band 12 to hold battery 28 in an area of wrist band 12 located directly opposite from housing 101. In this embodiment, wrist band 12 may include a flexible wiring harness disposed within an internally molded chamber that connects housing 101 with battery holder 105. In this manner, power from the battery 28 can be supplied to the electronics and sensor array located in housing 101. Via this channel and flexible wiring harness, a communication path can be established between 1) the sensors, microcontroller 40, transceiver 26, and any other electronic elements located in housing 101 and 2) any other electronics (e.g., a display unit or communication device, etc.) located remotely with respect to housing 101 along wrist band 12 (e.g., in battery holder 105).

Certain other embodiments may include batteries and corresponding battery holders spaced apart from one another. For example, in one embodiment, as shown in FIG. 3, a first battery (or battery bank) may be housed within battery holder 106 and, at the same time, another battery (or battery bank) could be housed within battery holder 105.

Data collection unit 10 can also be configured to include a cradle 108 that is either mounted to or integrated with battery holder 105, as shown in FIG. 3. Alternatively, cradle 108 can be mounted to or integrally formed with wrist band 12. Cradle 108 can be configured to receive and retain various items. For example, cradle 108 may be configured to provide one half of a standardized mating system such that components fitted with the other half of the mating system can be removably attached to cradle 108. Such components may include, e.g., watches, GPS units, heart rate monitors, general display units, or any other desired device. In certain embodiments, such units retained by cradle 108 may communicate with the sensors of data collection unit 10 (e.g., using a wiring harness routed within wrist band 12 or via a wireless communication path). In this manner, data from the sensors, either processed by the microprocessor 40 or unprocessed, could be collected, analyzed, and/or displayed by various units attached to cradle 108.

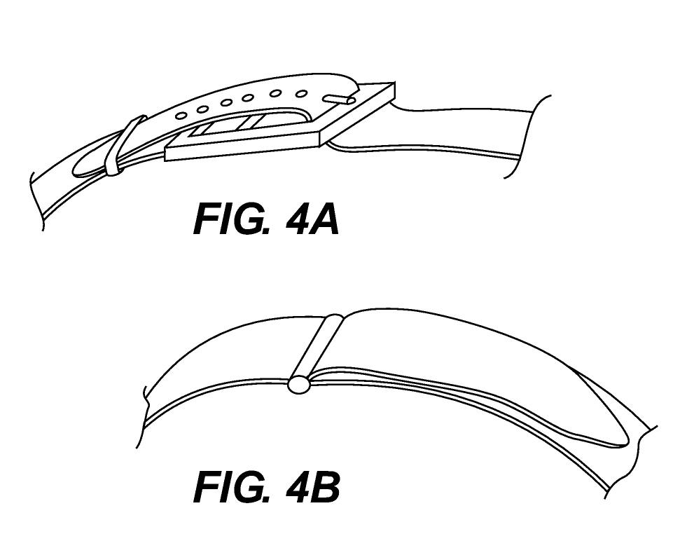

Data collection unit 10 may include any type of closure system suitable for securing data collection unit 10 to the wrist of a user. In one embodiment, for example, where the sensors, electronics, and/or batteries are not located on the underside of wrist strap 12, data collection unit 10 may employ a pin and hole type closure system shown in FIG. 4A. Data collection unit 10 may also include a hook and loop closure system as shown in FIG. 4B.

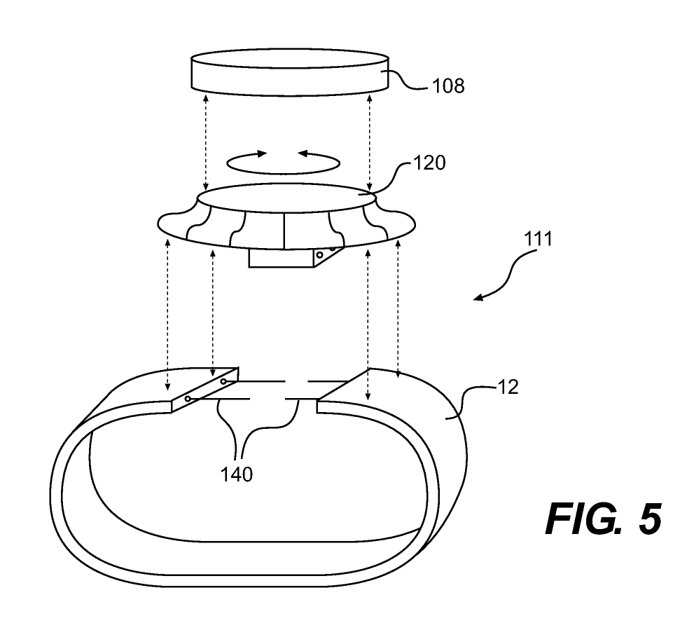

In other embodiments, data collection unit 10 may include a closure system 111, as shown in FIG. 5. In this embodiment, a wrist band 12 may include an opening near the top of the band. The opening may be configured to receive a closure member 120 that engages one or more tensioning elements 140. Closure member 120 may include an internal ratcheting mechanism that winds in or otherwise tightens tensioning elements 140 when closure member 120 is turned. Tightening tensioning elements 140 results in tightening of wrist band 12 against the wrist of the wearer. To release the tension on tensioning elements 140 and, thereby, loosen wrist band 12, closure member 120 may be turned in the opposite direction. Alternatively, or additionally, closure member 120 may include a release button that releases the internal ratcheting mechanism and allows tensioning elements 140 to loosen.

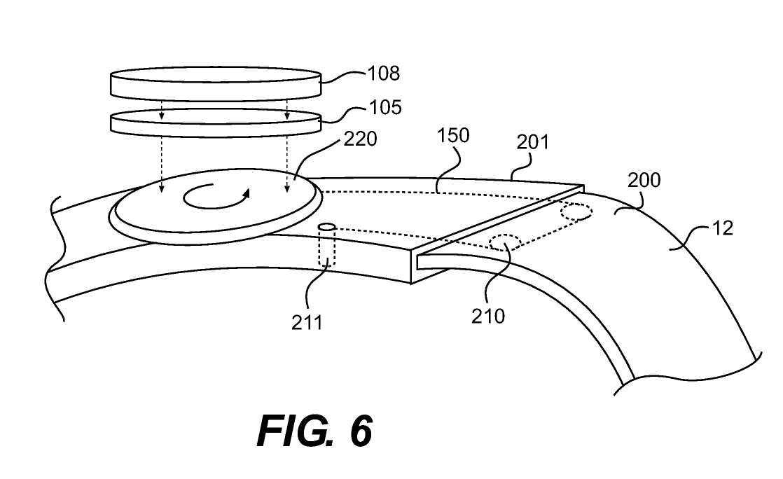

In another embodiment, data collection unit may include a closure system 112 fitted to wrist band 12, as shown in FIG. 6. Wrist band 12 may include a sheath configuration such that a portion 200 of wrist band 12 is configured to slide within a slightly thicker portion 201 of wrist band 12. Closure system 112 may include a dial wheel 220 that engages with tensioning elements 140. A tensioning element 150 may be internally routed through portion 201 of wrist band 12 such that it is led to retention pins 210 fixed within portion 200 of wrist band 12. Tension element 150 may be slideably attached to retention pins 210 and fixedly attached to an anchor 211 housed internal to portion 201 of wrist band 12. Closure system 112 may be configured such that turning of dial wheel 220 causes tensioning element 150 to wind around a spool (not shown) coupled to dial wheel 220. Winding of tensioning element 150 in one direction causes portion 200 of wrist band 12 to extend into portion 201, thereby tightening wrist band 12 about the user's wrist. Loosening may be accomplished by rotating dial wheel 220 in the opposite direction. In certain embodiments a cradle 108 and/or a battery holder 105, as described above, may be configured to attach to dial wheel 220.

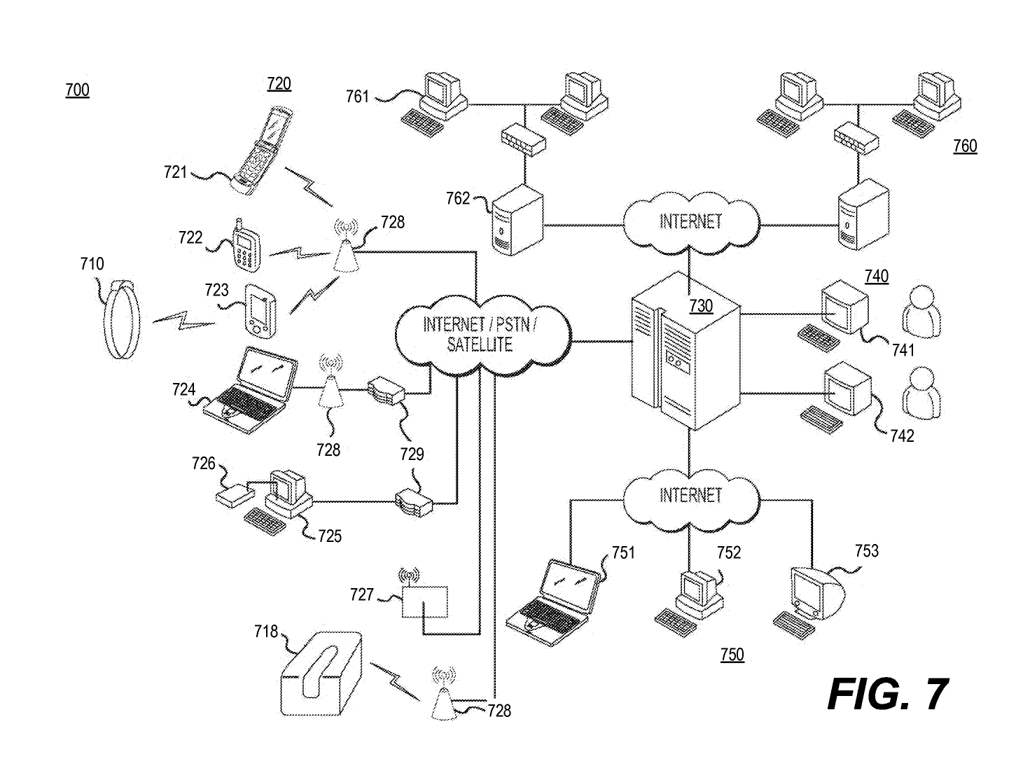

FIG. 7 provides a diagrammatic representation of a physical activity tracking and rewards allocation system 700 according to an exemplary disclosed embodiment. System 700 may include any suitable array of components for tracking the physical activity of one or more individuals, determining rewards based on the physical activity level of the one or more individuals, and allocating the rewards to the one or more individuals. In one embodiment, system 700 may include data collection portals 720, a mainframe 730, maintenance terminals 740, user nodes 750, and sponsor access nodes 760. Other embodiments of system 700 may include additional or alternative components where needed to provide any desired functionality for system 700.

System 700 may be configured to communicate and acquire data from one or more data collection units 710. Data collection units 710 may be worn by a user and may include at least one sensor for collecting data indicative of the physical activity level of the user. For example, data collection unit 710 may include a sensor array (including one or more sensors) configured to monitor biological markers that vary with the level of physical exertion of an individual. The monitored biological markers may include, for example, pulse rate, body temperature, physical movement, blood oxygen content, and/or any other suitable marker. Within the sensor array, each sensor may be configured to monitor only a single biological marker. Alternatively, an individual sensor in the array may be configured to monitor multiple biological markers.

Data collection units 710 may be configured to collect and store raw data collected from the sensor array. While it is possible to store raw data collected from the sensor array, a microcontroller on data collection units 710 may alternatively be configured to store processed data. For example, each data collection unit 710 may be configured to calculate pulse rate, pulse rate over time, oxygen content, physical movement, and/or temperature and average each calculated value over periods of up to thirty seconds, sixty seconds, or more to remove noise and enhance accuracy of the readings. The microcontroller can be configured to store these time averaged, filtered temperature/pulse rate/oxygen content/physical movement readings at preselected intervals (e.g., once or twice per minute). Such a scheme can conserve memory resources yet still provide useful information.

The data collected by data collection units 710, whether in raw form, time averaged filtered form, or in another processed format, can be transmitted or collected by system 700 via data collection portals 720. Data collection portals 720 may include any type of device suitably equipped for collecting data from data collection units 710. For example, data collection portals 720 may include a device cradle 718, a reader unit/pod 719, a cellular phone 721, a smart phone 722, a personal data assistant 723, a laptop computer 724, or other type of electronic device that can be configured to communicate with data collection units 710. In one embodiment, data collection portals 720 may be configured to communicate with data collection units 710 via a Bluetooth, wired, optical, or other type of data link. Data collection portals 720 may also include a personal portal 726 configured as a peripheral device to provide a computer 725, for example, with an ability to communicate with a data collection unit 710. Data collection portals 720 may also include a public portal 727. A public portal 727 may include a unit positioned in malls, public parks, fitness centers, sporting fields or any other public or private location frequented by users of data collection units 710.

In certain embodiments, data collection portal 720 may include a cradle unit 718 adapted to hold, or otherwise contact, the data collection unit 710. Such a cradle may facilitate the interrogation of data collection unit 710 and/or the transmission of data between data collection unit 710 and data collection portal 720. For example, in addition to a wireless connection between data collection unit 710 and cradle unit 718, data collection unit 710 and cradle unit 718 may communicate via an electrical pathway formed by physical contact between electrical connection points on data collection unit 710 and corresponding electrical connection pins on cradle unit 718. Cradle unit 718 may also be configured to recharge data collection unit 710.

Data transmission to data collection portals 720 may be initiated by either data collection units 710 or data collection portals 720. In one embodiment, data collection portals 720 may be configured to sense the in-range presence of a data collection unit and then initiate collection of data from data collection unit 710. Alternatively, or additionally, data collection unit 710 may be configured to detect the presence of an in-range data collection portal 720 and, in turn, initiate transmission of data to that portal.

In yet another embodiment, data collection portal 720 may be configured to emit an interrogation signal that, when received by a data collection unit 710, may prompt the data collection unit to transmit stored data to the data collection portal 720. For example, rather than data collection unit 710 periodically searching for a suitable data collection portal within range, data collection unit 710 may be configured to simply respond to an interrogation signal continuously or periodically emitted from a data collection portal 720. If data collection unit 710 receives such an interrogation and determines that the emitting data collection portal is within transmission range, then data collection unit 710 can activate a transceiver associated with the data collection unit 710 and commence data transmission.

Transmission between data collection units 710 and data collection portals 720 may be accomplished over any suitable transmission range. In certain embodiments, data collection unit 710 may be configured to transmit data to portals located within about ten feet of a data collection portal 720. In other embodiments, this transmission distance may be extended up to about 50 feet.

Moreover, data transmission may be accomplished via any suitable scheme for transmission of data. In one embodiment, the data stored in data collection unit 710 may be transferred to a data collection portal 720 via a wired connection including a cable and cable interface. Data transmission between data collection unit 710 and data collection portal 720 may also be accomplished via a wireless connection including a radio frequency or optical transmission link. In certain embodiments, for example, data collection unit 710 can be Bluetooth or Zigbee enabled or may transmit data to a data collection portal 720 via an infrared optical link.

When communication is established between data collection unit 710 and a data collection portal 720, download of the data stored on data collection unit 710 may commence, for example, after proper identification of the user and of the portal has been achieved. This may prevent eavesdropping by unauthorized parties. Identification of the user may include transmission of a unique code assigned to each data collection unit and/or user of the data collection unit. A user-selectable password can be used to allow data to be downloaded by the data collection portal.

In other embodiments, passive identification of a user may displace the need for password protected downloads. For example, data collection unit 710 may be configured to determine and store a biological signature of an authorized user of the data collection unit. Such a signature may be determined using the same array of sensors used monitor temperature, blood oxygen level, physical movement, and pulse rate. Alternatively, one or more additional sensors (e.g., a skin pigment sensor, pH sensor, etc.) may be included on data collection unit 710 to aid in user recognition.

Once transmission of data stored in data collection unit 710 commences, a handshaking process may be employed to validate the integrity of the data transmitted and to request retransmission of the data, if necessary. After the data collection unit establishes that the data has been successfully transmitted to the data collection portal, the microcontroller in data collection unit 710 can optionally delete the previously stored data.

Transmission of data to a data collection portal 720 may be controlled based on the availability of stored data. For example, if no new data has been stored in data collection unit 710 since the last successful download, then the microcontroller of data collection unit 710 may determine that there is nothing to transmit. Under these conditions, the data collection unit 710 may forego searching for a suitable data collection portal 720 and will remain powered down despite the presence of a detected in-range data collection portal 120.

Once a data collection portal 720 has received data from a data collection unit 710, that portal can store the data in a memory associated with the portal. Alternatively, or additionally, the receiving portal can simply forward the received data to a mainframe 730, which may be configured to operate as a core unit of system 700 by tracking the physical activity of individuals, allocating rewards, and obtaining scalable measurements of individual fitness.

The data received by data collection portals 720 can be transmitted to mainframe 730 by any suitable method and along any suitable communications path. Such communication paths may include wireless repeater units 728, routers 729, and any other communications equipment known in the art. In one embodiment, the data collection portals 720 can communicate with mainframe 730 via a wireless network (e.g., a cellular communications network), the Internet, satellite, public switched telephone network (PSTN), or any combination of these or other communications pathways.

Mainframe 730 may be configured to perform many tasks associated with system 700. For example, mainframe 730 can store and maintain user accounts (e.g., in storage area networks housing a database), process data associated with the physical activity level of individual users, calculate rewards based on the physical activity level of individual users, allocate rewards to user accounts based on the user's physical activity level, and generate or report a user's fitness profile. Mainframe 730 can also enable individual users to access their respective accounts, for example, to review physical activity data, review accrued rewards, monitor his or her fitness profile, and access any other features provided by system 700. Mainframe 730 may also compile selected data or data summaries and may provide access to this data and/or data summaries to selected entities, including corporate sponsors, health insurance providers, associations, the military, or any other entity that may have an interest in monitoring physical activity data.

Mainframe 730 may include a single server or may include multiple servers networked together. Mainframe 730 may also include power-outage back-up capabilities to secure continuous operation (24/7). Any number of devices may be included as part of or peripheral to mainframe 730. Such devices may include clustered World Wide Web servers, clustered database servers, storage area networks, fiber switches, firewalls, intrusion prevention systems, routers, switches, LTO tape drive, an LTO tape library, an APC InfrastruXure UPS System, and any other device or devices to provide a desired level of functionality. Mainframe 730 may be connected via Fibre Channel to the storage area networks that contain the user database. Connectivity to the Internet may be provided by Gigabit Ethernet connections to a network switch. There also may be redundant paths to the Internet provided by a local ISP using Cisco routers and T1 and/or DS3 connections.

A primary feature offered by physical activity tracking and rewards allocation system 700 is the ability to convert the physical activity level of a user into a “commercial value” or currency that the user can use to purchase various goods or services. In this way, the user may be motivated to exercise or otherwise maintain a particular level of physical activity in order to accrue currency for rewards redemption.

System 700 also offers the ability to use the physical activity of the user as a standard of measurement to determine an individual fitness profile, which is scalable for a unique but relative comparison with a broader demographic. Thus, third parties may use a uniform comparative measure of fitness to evaluate and monitor physical activity of one or more individuals and to compare individual fitness profiles to a selected broader demographic.

In one embodiment, the currency that can be used to acquire goods and services rewards may take the form of an electronically determined unit calculated based on the time spent in a predetermined physical activity zone or above a system determined individual predetermined threshold or baseline. Such currency may be referred to as activity units. Activity units may be allocated to an individual user account whenever the individual's physical activity pattern exceeds, by a predetermined amount, a stored baseline pattern associated with the individual. The rate at which the individual accrues activity units can be set at any suitable value. For example, in certain embodiments, one activity unit may be accrued for each minute that a user's physical activity level is maintained within a personal activity zone defined by a predetermined threshold above the individual user's baseline pattern. Of course, it is also possible for multiple activity units, or even less than one activity unit, to be awarded for each minute spent in the activity unit zone above the predetermined threshold.

System 700 can be configured to collect and store each user's baseline and zoned activity history and reflect this history in terms of minutes or hours spent within the baseline or zoned activity levels. The number of activity units accrued may be synonymous with the user's personal fitness progression and may be directly reflected by the individual's fitness profile. Because activity units may be directly related to time that an individual spends exercising, an individual's fitness level or physically active life style may also be associated with accrued activity units. In general, the more activity units that a user accrues, the greater the fitness level of that user will be. Further, as an individual accrues activity units, the individual's fitness profile will likely reflect a higher level of fitness and may compare more favorably to a broader demographic. Further, because the user may be motivated to accrue activity units as a currency that can be redeemed for various rewards, the user is essentially motivated to exercise and to achieve a greater fitness level.

Various programs may be instituted to encourage users to accumulate activity units. For example, a user's physical activity status may be categorized to reflect the rate at which the user accrues activity units. If zoned activity reaches up to 1,000 activity units in a 4-week period, for example, the individual may be categorized by system 100 as “moderately active.” Further, if zoned activity reaches more than 1,000 activity units in a four week period, then the user may be categorized as “active.” These categories may be reflected, for example, via the individual's fitness profile. Various additional levels or sub-levels may be assigned, as desired, to indicate a user's activity level progression or deterioration over time. In certain embodiments, the rate of activity units accrual may be tied to the user's physical activity status level. For example, a higher status level may translate into a different rate (e.g., a higher rate) of activity units accrual. Providing different rates of accrual for higher physical activity status levels may encourage individuals to move from lower physical activity status levels to higher ones.

Further, various forms of activity unit bonuses may be associated with physical activity status levels. For example, when a user moves to a more active status, a bonus may be awarded. Similarly, bonuses may be awarded for maintaining a certain physical activity status level over a certain period of time. In this way, a user may be motivated not only to exercise in order to accrue activity units currency, but the user may also be motivated to increase his or her physical activity status or to maintain a certain level of physical activity over an extended period of time in order to receive bonuses.

System 700 may be configured to allocate bonuses upon achieving certain milestones. For example, if a user accrues a certain number of activity units (e.g., 1000) within a preselected period of time (e.g., 4 weeks), then a bonus may be awarded to the user. The award of bonuses are not limited to the examples provided. Rather, bonuses may be awarded for any predetermined event relating to the physical activity level of a user.

Conversely, system 700 may also be configured to provide disincentives for becoming less physically active. For example, if a user moves to a lower physical activity status level, he or she may accumulate activity units at a different rate (e.g., more slowly) than at a higher status level. Further, system 700 may be configured to levy a penalty for moving to a lower physical activity status level from a higher status level. System 700 may be configured, however, to recognize potential causes for observed reductions in physical activity and forego penalties, where appropriate. For example, as an individual ages, his or her level of physical activity may decline as a natural part of the aging process. Additionally, an individual may become less physically active following a debilitating injury. Under such circumstances, system 700 may be configured to waive any penalties that would have otherwise been imposed in response to an observed reduction in physical activity.

The algorithm used to calculate activity units based on the recorded physical activity of a user may constitute a multi-part algorithm and may run on either data collection unit 710, on a server associated with mainframe 730, or partially on data collection unit 710 and partially on mainframe 730, or any other suitable computing device associated with system 700. In a first part of the algorithm, a user's physical activity level is monitored to determine whether that activity level qualifies as “zoned” activity for which activity units may be accrued. In a second part of the algorithm, the number of activity units to be awarded may be calculated based at least in part on time that a user spends in zoned physical activities.

In one embodiment, zoned physical activities may be determined based on a predetermined set of criteria applied uniformly to all users of a data collection unit 710. For example, a zoned physical activity may be defined as any activity that causes a measured physical parameter associated with an individual user to exceed a preselected threshold value. One such measured physical parameter may include a user's heart rate, for example. Microcontrollers associated with data collection units 710 may be configured to universally credit users with a zoned physical activity determination whenever the heart rate of those users exceeds a predetermined value (e.g., 110 beats per minute, or some other suitable heart beat threshold). In addition to heart rate, other physical parameters may be used, including, e.g., blood oxygen saturation value, body temperature, physical movement, or any combination of these or other suitable parameters.

In another embodiment, zoned physical activities may be determined according to the unique attributes of a particular user, rather than through application of a universally applied standard. In such an embodiment, the determination of a zoned physical activity may depend on a baseline fitness level for each individual. Such a baseline fitness level may be calculated by monitoring any suitable physical parameter, determining a value for that parameter associated with a resting condition for the user, and using the resting value of the physical parameter as a fitness level baseline unique to an individual. Suitable physical parameters for determining a resting condition of an individual may include, for example, heart rate, blood oxygen saturation level, body temperature, physical movement, or any combination of these or other suitable physical parameter values.

A baseline fitness level may also be determined according to an algorithm that depends on contributions from one or more physical parameter values. For example, an individual's baseline heart rate, IB₁, may be defined as the average of the lowest average heart rate (r) over a certain period of time (t) when the body temperature of the individual is stable. This baseline heart rate value may be represented as:

$${IB}_{1} = \frac{1}{T} {\int }_{0}^{T} r d t$$

An individual's baseline body temperature, IB₂, may be defined as an average of body temperature (f) over certain period of time (t) while the individual experiences his or her lowest average heart rate. The baseline body temperature may be represented as:

$${IB}_{2} = \frac{1}{T} {\int }_{0}^{T} f d t$$

An individual's baseline blood oxygen level, IB3, may be defined as the average blood oxygen level (b) over a certain period of time (t) while the individual experiences his or her lowest average heart rate. The baseline blood oxygen level may be represented as:

$${IB}_{3} = \frac{1}{T} {\int }_{0}^{T} b d t$$

Once an individual's baseline fitness level is determined by the method outlined above or by any other suitable method (e.g., by monitoring resting heart rate, among others), this baseline fitness level can be used to determine when the physical activity of a user qualifies as zoned physical activity. First, an average sensed vital signs quantity (ASVS) may be calculated based on the outputs of sensors that monitor a user's vital signs or other physical parameters. In the case of an array of sensors that monitor heart rate, blood oxygen level, and body temperature, the ASVS may be represented as:

ASVS=k₁×S₁+k₂×S₂+k₃×S₃

where S₁ represents current blood oxygen level, S₂ represents current heart rate, S₃ represents body temperature, and k₁, k₂, and k₃ are constants.

With the ASVS and the baseline fitness level, a physical activity score (PAS) may be calculated using the following relationship:

PAS=(k₁×S₁)/IB₁+(k₂×S₂)/IB₂+(k₃×S₃)/IB₃

If the PAS exceeds a certain predetermined threshold value, then the physical activity qualifies as zoned physical activity for which activity units may be accrued. Of course, PAS can be determined using any other suitable relationship. For example, an individual's PAS may depend solely on heart rate, any other sensed value, or any combination (weighted or otherwise) of sensed values.

Any portion of the algorithm can run on a data collection unit 710. In one embodiment, a microcontroller onboard at least one data collection unit 710 associated with system 700 may be configured to determine a baseline fitness level of an individual (using IB₁, IB₂, and/or IB₃, or via any other suitable method). The microcontroller may also be configured to calculate an ASVS based on the output of sensors included on data collection unit 710 and determine a PAS by comparing the ASVS to the PAS. The microcontroller can further be configured to monitor and store the total amount of time that the individual's PAS represents zoned physical activity (ZPA_{T}). In such an embodiment, the information transmitted from the data collection unit 710 to any of the data collection portals 120 could include ZPA_{T}. The transmitted data could also include data indicating the baseline fitness level of the user or any data associated with the individual user. Using this ZPA_{T} value, mainframe 730 could determine the amount of activity units that correspond to ZPA_{T} for the particular user. Alternatively, the microcontroller on data collection unit 710 could convert ZPA_{T} to activity units and forward this information to data collection portals 720.

In another embodiment, the microcontroller associated with a data collection unit 710 may be responsible for fewer calculations. In such an embodiment, the microcontroller may be configured to monitor outputs of sensors associated with the data collection unit 710, store these outputs as data, and transmit this data (either conditioned (e.g., by time averaging) or unconditioned) to a data collection portal 720 at regular intervals, when commanded by a user, or when data collection unit 710 is brought within a suitable communication range of a data collection portal 720. In this embodiment, mainframe 730, or another suitable computing device associated with system 700, would be responsible for determining the baseline fitness level of each user of a data collection unit 710; determining ASVS, PAS, and/or ZPA_{T} based on the data forwarded by the data collection unit 710; and determining the number of activity units to be allocated to the individual.

It is also possible that the microcontroller associated with a data collection unit 710 can perform an intermediate portion of the algorithm. In such an embodiment, the microcontroller may be responsible for calculating a baseline fitness level and transmitting that information to data collection portals 720 along with raw or conditioned data relating to the output of sensors included on data collection unit 710. Alternatively, the microcontroller could calculate ASVS, PAS, or ZPA_{T} and forward any of these quantities to data collection portals 710 with any other data relating to the physical activity of the individual.

Thus, system 700 may be configured such that mainframe 730 performs substantially all of the calculations associated with the algorithm and the microcontrollers of data collection units 710 forward the basic underlying data for those calculations. Alternatively, the individual microcontrollers of data collection units 710 can be configured to perform most, if not all, of the calculations associated with the algorithm and forward to mainframe 730 the results of those calculations. Further still, the calculations associated with the algorithm can be shared between mainframe 730 and the microcontrollers of data collection units 710 (or with any other computing device associated with system 700) in any desired proportion. It is even possible to have certain data collection units perform more of the algorithm than other data collection units. Mainframe 730 may be configured to accommodate differences in data provided by the various data collection units associated with system 700.

In the algorithm, the predetermined threshold against which the PAS is compared (i.e., to determine whether physical activity qualifies as zoned physical activity for which activity units may be accrued) may correspond to any desired threshold level. Setting the predetermined threshold lower, rather than higher, however, may minimize the risk of an individual overexerting himself in an attempt to accrue activity units. The purpose of the system or program is to encourage general fitness through moderate exercise. Overexertion can be dangerous. Individuals should be encouraged to exercise well within their physical limits and certainly well below the point of overexertion.

In one embodiment, the threshold (e.g., the IMAT: Individual Minimum Activity Threshold) used to compare against PAS may correspond to a value determined by a medical or health related board or association. Such an IMAT may correspond to moderate-intensity physical activity, such as any activity that requires about as much energy as walking two miles in 30 minutes. The IMAT may also be based, at least in part, on heart rate. For example, the IMAT may correspond to the individual's target heart rate for moderate-intensity physical activity. Such a heart rate value may correspond to about 50% to about 70% of his or her maximum heart rate, which may be based on the age of the individual. For example, an estimate of a person's maximum age-related heart rate can be obtained by subtracting the person's age from 220. Thus, a 50-year-old person has an estimated maximum age-related heart rate of about 170 beats per minute (bpm) (i.e., 220-50). The 50% and 70% levels would be:

50% level: 170×0.50=85 bpm, and

70% level: 170×0.70=119 bpm.

Thus, to encourage moderate-intensity physical activity for a 50-year-old person, the IMAT may be set as a value from about 85 bpm to about 119 bpm.

In another embodiment, the IMAT may be associated with a certain metabolic equivalent level used to measure physical activity intensity. For example, the level of effort expended during a physical activity can be represented in terms of a metabolic equivalent (MET). Such a unit may be used to estimate the amount of oxygen used by the body during physical activity. The energy (or oxygen) required for a body to read a book, for example, may equal 1 MET. In such an embodiment, the IMAT may be set somewhere between about 3 and about 6 METs, which may correspond to a moderate-intensity level.

To encourage general overall fitness of individuals through physical activity, system 700 allocates activity units (i.e., a currency) which can be redeemed for rewards. Such rewards can be monetary. Alternatively or additionally, such rewards may include free or discounted merchandise (e.g., clothes, sporting equipment, airline tickets, food, concert tickets, among many others) or free or discounted services from a sponsoring entity (e.g., hotel visits, spa services, fitness evaluation testing, deductible payments for doctor visits, among many others). Thus, an individual's collected (or earned) activity units represent an individually earned currency or value based on physical activity, as these activity units can be redeemed against commercially available products and services.

As system 700 calculates and awards activity units to an individual user, system 700 updates an account for that individual and adds the newly accrued activity units. Each individual user of a data collection unit 710 may have a unique account in which the activity units accrued and redeemed by the individual can be tracked. Account information may be stored in one or more databases associated with mainframe 730.

System 700 may require maintenance from time to time. For this purpose, system 700 may include one or more internal access nodes 740 to provide system administrators with access to the databases, applications, user data, etc. of system 700. In one embodiment, these internal access nodes 740 include terminals 741, 742 in communication with mainframe 730.

Individuals can access their accounts in any suitable manner. For example, data collection portals 720 may be equipped with a user interface that allows an individual to access his or her account. Additionally, individuals may be able to access account information via user nodes 750. Such user nodes may include, for example, a laptop computer 751, a PC 752, terminal 753, a hand-held device (not shown), or any other device suitable for accessing information. While user nodes 750 are depicted in FIG. 1 as being in communication with mainframe 730 via the Internet (e.g., via a Web-based browser application), any other suitable communications scheme may be employed. Further, in embodiments where data collection units 710 include a display, such data collection units may be configured to allow an individual to view account data on the display. Such access could provide real-time information, such as whether the IMAT has been exceeded, the rate of activity units accrual, the account balance, or any other desired information.

With access to account information, an individual user can determine his or her activity unit balance or review account activity (e.g., activity unit credits or debits corresponding to reward redemption activities, among other account activities). The individual may also print a rewards redemption certificate or coupon, redeem activity units for rewards via an electronic transaction (e.g., by using accrued activity units to make a purchase from an online retailer), change passwords and other administrative tasks, or perform any other account-related activity. System 700 may also be configured to provide an individual's historical activity both in numbers and in graphical form for both accumulated activity units (Activity Histograms) and transacted/redeemed units (e.g., a report of when, where, and how many activity units were redeemed and what product, service, or company, etc. was involved in the transaction). Individual account statements can be produced, printed, and mailed via post and/or e-mail to each individual on a regular basis. Updated statements can also be printed by a user at any time by accessing his or her own individual user account profile and printing locally. These certificates can be used, for example, as evidence of or as a profile reflecting an individual's active lifestyle pattern and/or fitness level progression and as a way of increasing the person's perceived fitness value to a medical entity, insurance provider, employer, the military, or any other institution that values good health and active life styles as essential components to advocating positive social change. Individual users of system 700 may also be e-mailed periodically with special offers. Such offers may include an offer to accrue activity units at a greater rate during a certain limited time period. Such offers may also include access to certain products or services previously unavailable or to products and services at a discounted rate. Such offers may also be associated with observed holidays.

As individual activity unit balances increase, each user may enjoy a higher level of credit expendability and status in the program. E-mail alerts can be sent to update the user about his or her progress and the user's server profile may be updated to reflect user progression.

In certain embodiments, system 700 may also provide access to one or more corporate sponsors, corporations, insurance companies, charitable associations, or other entities. Such access may be achieved via sponsor access nodes 760, which may include one or more computers 761, a server 762, or any other components or devices for providing a communication path (e.g., using the Internet) to mainframe 730.

Such entities may wish to have access to system 700 for various reasons. For example, corporations that utilize data collection units for some portion of their employees may create an accounting principle to record the company's physical activity count (PAC). Such a measure can be recorded, for example, for use in negotiating lower health insurance costs or other employer-related benefits.

Entities (e.g., corporations, military, government, associations, or other groups) may also access system 700 to evaluate the fitness level of a particular individual or a group of individuals. For example, these entities may access and evaluate the fitness profile of a particular individual. Alternatively or additionally, these entities may access and analyze the fitness profiles of multiple individuals using, for example, a batch processing algorithm to assess the average fitness level of a selected group of individuals. These evaluations may be used, for example, to determine an overall fitness level for one or more particular individuals, employees, troops, members of an organization, etc. Among other uses, this information may be used to verify compliance with fitness regulations or goals, to negotiate reduced health insurance premiums, or to obtain subsidies, e.g., from the government or private sponsors, in exchange for maintaining a desired average fitness level among a certain population of individuals.

A user fitness profile may include any desired information relating to the fitness or physical activities of an individual. In one embodiment, the fitness profile may be configured to reflect the number of activity units accrued by the individual, an elapsed time spent participating in zoned physical activities (e.g., total elapsed time, average time per month, week, and/or day, or an amount of time over a selected time period), a fitness score or qualifier indicative of the general fitness level of the individual (based, for example, on a predetermined algorithm or set of criteria), a trend in fitness level, time spent as a participant in the system or program, and any other desired information relating to the fitness of an individual. Fitness profiles may also include information relating to vital statistics associated with an individual including, for example, heart rate data, blood oxygen saturation data, body temperature data, and/or physical movement. In addition to individual-specific fitness profiles, system 100 may also be configured to determine/maintain a fitness profile for a group of individuals (e.g., workers of a common entity, residents of a particular jurisdiction, members of a club or group, military units, etc.).

After acquiring a data collection unit 710 and prior to commencing with the data collection and rewards allocation process, initial registration with system 700 may be performed. This initial registration process may be accomplished by an individual user accessing a website to register a new membership and create a user profile for his or her account. The individual may also provide data to system 700, which may be maintained with the individual's user account. This data may include, among other things, the individual's name, a system password, bracelet ID, telephone number, emergency contact (and contact number), age, sex, geographic location, address, e-mail address, activity preference, other interests, training schedule, upcoming events, reference to personal website, etc. Personal medical data can also be entered in the designated server profile and downloaded to the data collection unit 710 associated with a particular user. This information could potentially be retrieved in an emergency situation by EMT personnel and may include blood type, allergy information, pre-existing conditions such as diabetes level, and emergency contact numbers.

The initial registration process may also include a data collection unit calibration process. This calibration process may begin by powering on the data collection unit and entering a unique PIN for the data collection unit. The PIN enables a system 700, including data collection portals 720 and/or mainframe 730, to recognize each data collection unit 710. PIN verification may be made regularly by server maintenance staff, i.e. once per quarter or semi-annually. It should be noted that this PIN is separate from a PIN that a user may establish to restrict access to the user's account on mainframe 730. Further, rather than entering a PIN manually, data collection unit 710 may be configured to automatically transmit its serial number or other PIN to a data collection portal 720 and, therefore, to mainframe 730 for verification purposes.

Next, data collection unit 710, either together with other components of system 700 or on its own, may proceed with creation of an initial physical activity baseline for the individual. This portion of the calibration process would require the user to wear the data collection unit for a predetermined minimum amount of time (e.g., 24 hours or other suitable period of time) in order to establish a fitness baseline. Once the initial threshold and/or baseline is established, the data collection unit is ready to collect physical activity data. An indicator light, display, or other type of indicator can be used to alert the user when a suitable fitness baseline has been achieved and the data collection unit is ready for normal operation.

System 700 can be configured to automatically recalibrate data collection unit 710 on a periodic basis. For example, a new baseline fitness level may be determined by each data collection unit 710 after a certain amount of time has passed (e.g., weekly, monthly, or at any other desired interval) or whenever a certain amount of zoned physical activity has been measured (e.g., after 20 hours or any other desired amount of zoned physical activity has been observed). Alternatively, this recalibration process could be configured to occur on a continuous basis. That is, as system 700 acquires data, the baseline fitness level of a user could be continually updated to reflect the most current fitness level for that individual.

Certain regulations may be instituted regarding the availability of activity units for redemption of rewards. In general, however, activity units are simply accrued in each user's individual account and can be redeemed at any point in time against member/sponsor companies' products and services. Each member company may determine what it would like to offer in exchange for a certain number of activity units. Each member company or government institution may also determine the period of time that its offer (discount or credit) is commercially valid (e.g., for 30 days or up to a year or more). In other words, some companies may have a more or less aggressive offering than others, both in terms of value and time.

The redemption process can be performed either electronically or in person. For example, a user may access an online website of a sponsor company or entity where certain products may be procured at least in part through redemption of activity units. Additionally, vouchers or coupons may be printed and presented to a corporate supplier or other entity for redemption in a traditional “bricks and mortar” retail setting.

Redemption may be made through a reward program or other website for any products or services offered through that site. Additionally, redemption may be made in person or through the website of any sponsoring corporation or entity that offers products or services through its own retail outlets (e.g., electronic or traditional stores). Further still, it is envisioned that redemption may occur at the retail outlets of non-sponsoring corporations that sell the products or services of sponsoring corporations or entities. For example, activity units could be used to purchase a bicycle made by a program-sponsoring bicycle manufacturer even when the bicycle is sold by a retail store with, perhaps, no affiliation with the program.

System 700 may be configured to provide a host of other features. For example, system 700 may be configured to verify individual fitness center attendance to a program enabled fitness center. System 700 may also be configured to incorporate and utilize GPS data. Such information may be used to enable individual location tracking or collection of geographical location information for mapping, routing, and planning purposes. In one embodiment, data collection unit 710 may incorporate a GPS capability to acquire and store specific cycling or running routes that can later be accessed and printed via a user profile and/or shared with other users registered with the program.

Given the data collection unit's multi-functional sensing and registration capabilities, other data may be collected, stored and transferred to/from mainframe 730. Such data may include, for example, athletic event timing information, such as start times, split times, and finishing times (or any other measure of individual timing performance) for running, walking, cycling, skiing, and triathlon events, among others.

The data collection unit may also function as an individual verifier and method of payment for individual entry to affiliated (designated) partner programs' facilities or service offerings. For example, a data collection unit may be configured to operate at least partially as an automatic debit system in which a user can automatically access an accumulated activity units simply by entering or establishing a communication link with a program sponsoring entity. In this way, a data collection unit could be used much like a debit card to access the user's accrued activity units balance rather than cash. A data collection unit may also be configured to allow an event participant to use accrued activity units as payment for registering for such events.

System 700 may also be configured to include user groups and other community features. Such features may include services, such as online advertising, news and promotional sharing, personal/social networking, event and sports promotion, sporting results, e-mails, blogs etc. System 100 may also include chat rooms or other public communications forums.

In general, system 700 may provide a convergent marketplace between individual users, the broader community, and sponsoring companies/organizations as a way of encouraging more active and healthy life styles through physical fitness. Consequently, the program community may include any group affiliated with an active lifestyle. Such groups may include those affiliated with individual sports, such as walking, running, cycling, skiing, swimming, triathlons, golf and tennis, or team sports, such as football/soccer, baseball, basketball, volley ball, ice hockey, etc. Route information and other special interest information may be shared among users of system 100. Such information may be even more readily available where system 100 includes a GPS capability.

System 700 could also be used as a service center to help communicate local, regional, national, and/or international information to the various users. Such information may include, for example, information relating to planned walks, runs, cycling events or other athletic/cultural or community-based activities that promote physical fitness and/or healthy/charitable lifestyles. System 700 may also offer information about local/regional/national member gyms, fitness and health clubs, or sports rehabilitation medicine or physical therapy facilities as a way of encouraging more people towards sanctioned programs at these facilities.

System 700 may be configured to provide bonuses for individuals competing or participating in certain sanctioned events. System 700 can also be configured to maintain an events database and store information relating to these events for later access. This way, individuals may be able to look up their events history and keep track of past performances across various sporting activities while earning authorized bonuses for participating in such events.

System 700 may be equipped with several fraud detection and/or prevention safeguards. For example, each data collection unit 710 may be provided with a unique serial number that can be regularly verified by mainframe 730. System 700 may require a user ID and password for access to user account information. System 700 may be configured to recognize unusual or “out-of-range” data that may have been fraudulently generated. System 700 may also be configured to determine a bio signature for an individual user based on outside temperature and one or more of the user's body temperature, blood oxygen level, physical movements, and heart rate information, for example. By recording a history for these values, or by monitoring other criteria, system 700 may be able to detect whether certain measured values or average values are outside of expected ranges for a particular individual. For example, if a 65 year old individual generates heart rate readings consistently above 190 beats per minute over a certain period of time, and historical data does not show such a high heart rate from past use of the device, system 700 may flag this account as potentially including fraudulently generated data. Under such circumstances, system 700 may generate an automated message requesting that the user explain the circumstances surrounding the physical activity during which the suspect data was acquired. System 700 may also be configured to forego an award of activity units upon detection of suspected fraudulent activity.

Based on data collected by data collection unit 10, 710, the disclosed system may also be configured to determine a type of activity in which the individual is or has engaged. Such a determination may be made, for example, using algorithms operating on microcontroller 40 of data collection unit 10. Alternatively, or additionally, such a determination may be made in mainframe 730 of system 700, as shown in FIG. 7.

As previously noted, data collection unit 10 may include an accelerometer 24 to monitor motion of data collection unit 10. In certain embodiments, accelerometer 24 includes only a single axis accelerometer configured to detect motion along one axis. Other embodiments, however, may include multiple accelerometers. In one exemplary embodiment, accelerometer 24 may include a three-axis accelerometer, which includes three accelerometers arranged orthogonally with respect to one another. With such an arrangement, accelerometer 24 may be able to detect or monitor movements along three separate axes.

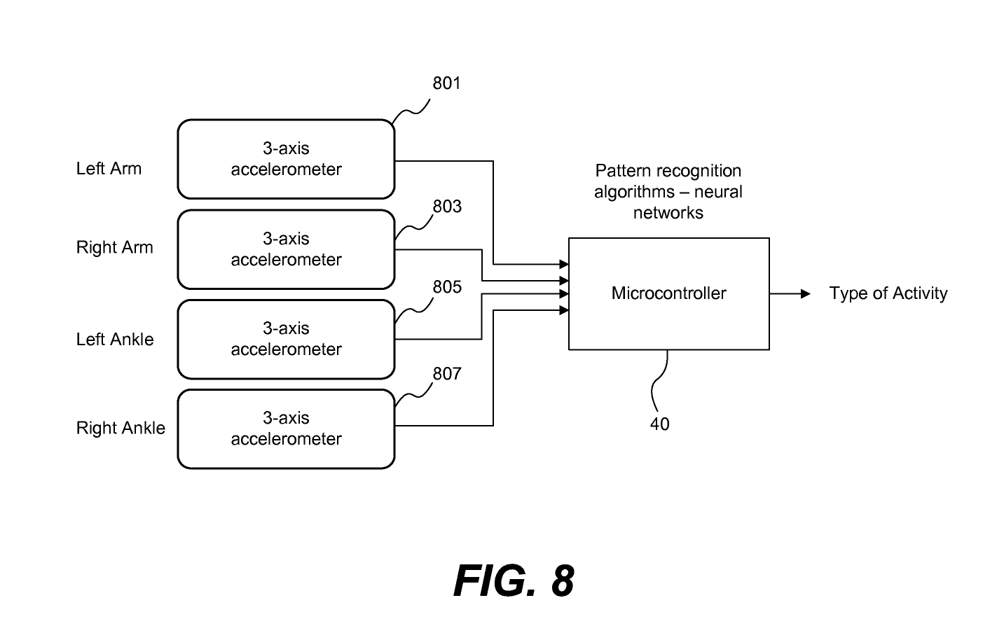

In addition to accelerometer 24 included in data collection unit 10, or data collection unit 710, other accelerometers 801, 803, 805, and/or 807 (as shown in FIG. 8) may be employed. Along with accelerometer 24 included in data collection unit 10, accelerometers 801, 803, 805, and/or 807 may be useful for the detection of movements associated with exercise and certain types of physical activity. Together, these accelerometers, or any subset thereof, can help confirm whether the wearer of data collection unit 10 is engaged in physical activity, can increase the accuracy of activity/inactivity-based measurements, and, can help determine the type of activity in which the wearer is engaged.

Accelerometers 801, 803, 805, and/or 807 may communicate with data collection unit 10 through any suitable method. In one embodiment, for example, the output of accelerometers 801, 803, 805, and/or 807 may be supplied directly or indirectly to microprocessor 40 of data collection unit 10. The output of these accelerometers, along with the output of accelerometer 24, may enable data collection unit 10 to determine the type of activity in which an individual is engaged. Alternatively, or additionally, information associated with the output of these accelerometers (e.g., the outputs themselves or processed data relating to the outputs) may be provided to system 700 for processing and activity determination.

In general, accelerometers, such as accelerometers 801, 803, 805, and/or 807 provide a response to an acceleration (change in velocity). A linear accelerometer (1-axis) produces a response when the acceleration has a component in the same axis as that of the accelerometer. A 2-axis accelerometer produces independent responses in a 2-axis surface, such that it can determine the direction of the acceleration in a surface. A 3-axis accelerometer provides a complete representation of the acceleration in a three-dimensional space.

The indication provided by the accelerometer is proportional to the acceleration to which it is being exposed. A mathematical integration of the acceleration results in an indication of velocity. A second mathematical integration provides an indication of displacement. On the other hand, the mathematical derivative of the acceleration provides an indication of shock.

The combination of all these measurements can be used to determine the type of activity being performed by an individual. For example, certain activities may be associated with a certain set of characteristics that may be observed based on analysis of the outputs of accelerometers 801, 803, 805, 807, and/or accelerometer 24. For example: walking, jogging, and running produce a periodic acceleration when measured in the lower extremities, while exhibiting a shock component every time contact is made with the ground. The acceleration immediately following the detection of the shock can be used to estimate the speed of movement, which when coupled with the time between successive shocks can be used to estimate the distance traversed. The numerical integration of the distance traversed between successive shocks can then be used to estimate the total distance traversed by a person. Furthermore, indications from accelerometers placed in the upper extremities can be correlated with those of the lower extremities to further validate the periodic movement of the arms associated with walking, jogging, and running.

Tennis, racquetball, and other racquet-based sports provide a different shock signature. In addition to the shock exhibited by the lower extremities, one of the accelerometers in the upper extremities will also detect a shock component every time the racquet makes contact with the ball. Occasionally, as when doing a backhand swing using both arms, the shock component will appear on accelerometers on both left and right arms. Similar analysis can be done for sports such as swimming (style-dependent), bicycling, soccer, football, ping-pong, skiing, walking, parachuting, horseback riding, etc.

Determination of the type of the physical activity may be based on the interpretation of data provided by the accelerometers. Accuracy of the determination of the type of activity may be increased through use of multiple accelerometers. For example, use of accelerometer 801 along with accelerometer 24 may provide a greater accuracy in activity determination than, e.g., using accelerometer 24 alone. In some embodiments, the accuracy of this determination may be even greater through use of additional accelerometers, such as accelerometers 803, 805 and/or 807. While in certain embodiments, an activity type determination could be accomplished with only one accelerometer, two or more accelerometers may provide a more accurate determination. It should be noted that the accuracy of the activity type determination could be hindered by various factors (e.g., if a right handed person wears an accelerometer on the left arm and the shock component associated with certain activity goes at least partially unobserved).

The determination of the type of physical activity may also be based on the output provided by other sensors. Such sensors may include infrared sensors and/or temperature sensors. Information from these sensors relating to blood oxygen content, heart rate, body temperature, etc. may be associated with certain activity types and, therefore, may be used alone or in conjunction with output from one or more accelerometers to identify a type of physical activity of a user.

The activity type analysis can be performed using artificial intelligence based on a pattern recognition algorithm implemented using neural networks. The data from the accelerometers may be mathematically analyzed to provide speed, displacement, and shock information to the neural network, which may then process the information to find the best match with known activity type signature patterns.

The operation of the pattern recognition algorithm may be based on training of the neural network based on actual acceleration data obtained from performing a plurality of sport activities. The neural network may then associate a typical signature (when using a single sensor), or multiple signatures (when using more than one acceleration sensor), with a defined sport activity. The accuracy of the neural network may increase as the number of sensors increases and as sensors are placed on various parts of the body. Once a collection of sport activities has been obtained, the neural sensor network may be ready to operate autonomously and evaluate the type of activity being performed. The neural network does not need to be trained for every specific user, only for those types of physical activity for which there may be a need or desire to detect or otherwise make a determination of physical activity type.

The accelerometers use low power and can be self-contained with their own coin-sized battery. Communication with data collection unit 10, 710 can be accomplished through low power RF, for example, where no FCC permits are required. These communications can be encoded to minimize or prevent interference with other users. Possible implementations of accelerometers 801, 803, 805, an/or 807 may include mini-chips that could be attached to shoes (for the lower extremities), pants legs, socks, a simple band for one or both of the arms, sleeves of a shirt or jersey, wrist bands, watches, heart rate monitors, etc.

As described above, the presently disclosed systems enable determination of various parameter values associated with the activities of an individual. For example, data collection unit (with microcontroller 40), system 100, and/or system 700 can determine such parameter values as a particular activity type (e.g., running, swimming, cycling, tennis or other racquet sports, bowling, and many other sports or activities that involve general movement patterns providing a “signature” of the activity); time spent engaged in the activity; and/or exertion level while involved in the activity (e.g., both peak and average exertion levels). Based on this information, system 100, 700 may be configured to direct advertising to the individual tailored to the activities of the individual. For example, advertisements for running equipment (e.g., shoes, clothing, heart rate monitors, run tracking software, etc.) may be provided to the individual if the system determines that the individual's physical activities include running. These advertisements can be further tailored based on factors such as how long the individual spends engaged in running activities (e.g., frequency of engaging in the activity, time spent per activity session, and/or total time participating in the activity in a predetermined time period) and the individual's level of exertion during exercise. For example, an individual determined to be a high mileage, high-exertion level runner may constitute a higher valued subject for advertising resources, as such an individual may be likely to purchase more running-related equipment in general and may be more likely to replace equipment more often than, for example, a lower mileage runner or one that runs more infrequently or at lower exertion levels. Similar determinations for directing advertising resources can also be made based on any other recognized physical activity type.

It will be apparent to those skilled in the art that various modifications and variations can be made in the disclosed sensor unit without departing from the scope of the disclosure. Other embodiments of the disclosed systems and methods will be apparent to those skilled in the art from consideration of the specification and practice of the disclosure disclosed herein.

What is claimed is:

1. A physical activity data collection system, including:
one or more accelerometers configured to provide an output indicative of movements of an individual; and
a controller configured to:
analyze information relating to the movements of the individual, based on the output of the one or more accelerometers;
determine a physical activity type associated with the analyzed information; and
store the determined physical activity type in a memory.

2. The physical activity data collection system of claim 1 wherein the one or more accelerometers are included in at least one accelerometer unit in communication with a data collection unit that includes the controller.

3. The physical activity data collection system of claim 2 wherein the at least one accelerometer unit is configured to be worn on an extremity of the individual.

4. The physical activity data collection system of claim 2 wherein the data collection unit is configured to be worn on a wrist of the individual.

5. The physical activity data collection system of claim 1 wherein the one or more accelerometers are included in at least one accelerometer unit in communication with a data collection unit remotely located from the controller.

6. The physical activity data collection system of claim 5 wherein the data collection unit includes a processor configured to collect data from the one or more accelerometers, process the data, and send the processed data to the controller.

7. The physical activity data collection system of claim 1 wherein the controller is further configured to determine a duration associated with the type of physical activity in which the individual engages.

8. The physical activity data collection system of claim 1 wherein the controller is further configured to determine an intensity level associated with the type of physical activity in which the individual engages.

9. The physical activity data collection system of claim 1 further including:
one or more infrared sensors configured to provide an output indicative of a pulse rate of a individual; wherein the controller is configured to:
determine a pulse rate of the individual based on outputs from the one or more infrared sensors; and
determine the physical activity type based, at least in part, on the determined pulse rate.

10. The physical activity data collection system of claim 1 further including:
at least one temperature sensor configured to provide an output indicative of at least a body temperature of the individual;
wherein the controller is configured to:
determine a temperature associated with the body of the individual based on outputs from the at least one temperature sensor; and
determine the physical activity type based, at least in part, on the determined temperature.

11. The physical activity data collection system of claim 1 wherein the controller is further configured to cause one or more advertisements to be provided to the individual based on the determined physical activity type.

12. The physical activity data collection system of claim 7 wherein the controller is further configured to cause one or more advertisements to be provided to the individual based on the determined duration associated with the type of physical activity in which the individual engages.

13. The physical activity data collection system of claim 8 wherein the controller is further configured to cause one or more advertisements to be provided to the individual based on the determined intensity associated with the type of physical activity in which the individual engages.

14. A method of identifying a type of physical activity in which an individual engages, including:
monitoring outputs of one or more accelerometers configured to provide an output indicative of movements of the individual;
analyzing information relating to the movements of the user, based on the output of the one or more accelerometers;
determining a physical activity type associated with the analyzed information; and
storing the determined physical activity type in a memory.

15. The method of claim 14, further including causing one or more advertisements to be provided to the individual based on the determined physical activity type.
